(19) United States
(12) Patent Application Publication
(10) Pub. No.: US 2024/0091293 A1
(43) Pub. Date: Mar. 21, 2024

(54) IMMUNITY-ENHANCING COMPOSITION CONTAINING SYNEILESIS PALMATA EXTRACT AS ACTIVE INGREDIENT

(71) Applicant: Andong National University Industry-Academic Cooperation Foundation, Andong-si (KR)

(72) Inventor: Jin-Boo Jeong, Andong-si (KR)

(73) Assignee: Andong National University Industry-Academic Cooperation Foundation, Andong-si (KR)

(21) Appl. No.: 18/171,670

(22) Filed: Feb. 21, 2023

(30) Foreign Application Priority Data
Sep. 15, 2022 (KR) 10-2022-0116376

(51) Int. Cl. A61K 36/28; A23K 10/30; A23L 33/105; A61P 37/04

(52) U.S. Cl. CPC A61K 36/28; A23K 10/30; A23L 33/105; A61P 37/04

(57) ABSTRACT

Proposed is an immunity-enhancing composition containing a Syneilesis palmata extract as an active ingredient. The Syneilesis palmata extract activates macrophage phagocytosis, promotes the production of nitric oxide (NO), inducible nitric oxide synthase (iNOS), interleukin-1β (IL-1β), and tumor necrosis factor-α (TNF-α), and induces TLR2/4 stimulation. In addition, the Syneilesis palmata extract exhibits immunity-enhancing activity by activating macrophage autophagy, suggesting that the Syneilesis palmata extract can be applied to a variety of health supplement foods.

### STATEMENT REGARDING GOVERNMENT SPONSORED RESEARCH

This invention was made with Korean government support under “Smart Forest management innovative growth technology development and spread-smart forest income generation technology development” awarded by Korea Forestry Promotion Institute.

### CROSS REFERENCE TO RELATED APPLICATION

The present application claims priority to Korean Patent Application No. 10-2022-0116376, filed Sep. 15, 2022, the entire contents of which is incorporated herein for all purposes by this reference.

### BACKGROUND OF THE DISCLOSURE

### Field of the Disclosure

The present disclosure relates to an immunity-enhancing composition including a Syneilesis palmata extract as an active ingredient.

### Description of the Related Art

Even though humans are exposed to various pathogenic microorganisms and viruses during their lifetime, the host immune system protects the human body from these attacks. The innate immune response is the first defense system that protects system that protects the human body from harmful foreign pathogens, such as pathogenic microorganisms and viruses. Among immune cells responsible for the innate immune response, macrophages are representative immune cells that exhibit antigen presentation functions and phagocytosis against pathogenic microorganisms or viruses. Such activities of macrophages are enhanced by pathogenic microorganisms or viruses as well as immunity-enhancing factors, such as interferon (IFN). Activated macrophages secrete immunostimulatory factors, such as nitric oxide (NO), inducible nitric oxide synthase (iNOS), interleukin-1β (IL-1β, tumor necrosis factor-α (TNF-α), and the like, that enhance phagocytosis against pathogenic microorganisms of macrophages and infection sources, such as cytotoxicity against cancer cells and viruses. FurtheLmore, such immunostimulatory factors secreted from activated macrophages enhance the activities of helper T cells and natural killer cells (NK cells) and promote B cell maturation and clonal expansion, thereby playing a key role in early immune functions.

Therefore, induction of macrophage activation is widely known for strengthening both the innate and acquired immune systems, which contributes to the enhancement of human immunity. Toll-like receptors (TLRs) of macrophages recognize foreign pathogens and induce the activation of host-defense mechanisms. Activation of the TLRs is known to activate both the innate and acquired immune responses. Among the TLRs, TLR2/4 is known as the main target capable of enhancing the immune functions of patients with immune disorders, and drugs that stimulate TLR2/4 are clinically used in practice.

Recent studies have reported that autophagy can promote T cell responses by regulating the functions of antigen-presenting cells and T cells. In particular, macrophage autophagy activation via TLR2/4 stimulation has been reported to enhance innate and acquired immune responses by promoting macrophage antigen processing and presentation. In autophagy, microtubule-associated protein 1A/1B-light chain 3 (LC3) is used to investigate autophagosomes and autophagic activity and is responsible for the recruitment of cargoes to the autophagosomes. LC3-II formed from LC3-I is localized to the autophagosomes, so the amount of LC3-II is known to be directly proportional to the number of autophagosomes and autophagy-related structures. p62/sequestosome 1 (SQSTM1) is a selective autophagy receptor, and plays an important role in the delivery of various ubiquitinated cargoes to the autophagic pathway. Therefore, an increase in LC3-II and p62/SQSTM1 is known as a key indicator of autophagy.

In many cases, when a disease occurs in the body, immune functions are often compromised. Thus, substances capable of enhancing immunocompetence in cancer patients or the elderly are required. A variety of therapeutic agents and supplements have been developed and marketed so far to enhance immunity. However, due to the side effects caused by long-term use, the use of relatively safe supplements, rather than therapeutic agents, has been proposed as a desirable direction. Substances capable of enhancing immunity can be obtained from chemical compounds or compounds derived from animals or plants. However, in the case of chemical compounds, pharmacological mechanisms are diverse, or side effects may occur due to toxicity. In addition, when medicine raw materials are obtained from animals, toxic substances that cause zoonotic viruses or rare intractable diseases may be found. Thus, the risk of side effects is pointed out as a problem. In addition, when using plants rather than obtaining materials using chemical compounds or animals, there are advantages in terms of speed and costs. Thus, demand for developing natural immunity-enhancing substances derived from plants without side effects is growing.

Syneilesis palmata is a perennial herbaceous plant belonging to the Asteraceae family and has been used as a traditional medicinal plant and vegetable for a long time in Korea. Leaves and flowers of Syneilesis palmata have been used to treat pain, arthritis, gout, back pain, and bruises, and to improve blood circulation. Syneilesis palmata has been reported to have anti-inflammatory activity, anti-cancer activity, and anti-HIV-1 activity through various studies.

Hence, while studying a variety of physiological activities of a Syneilesis palmata extract, the inventors of the present disclosure have confirmed the immunity-enhancing effects of the Syneilesis palmata extract, and thus completed the present disclosure.

### DOCUMENT OF RELATED ART

### Patent Document

(Patent Document 0001) Korean Patent No. 10-1732365 (Title of the disclosure: PHARMACEUTICAL COMPOSITION FOR PREVENTING OR TREATING INFLAMMATORY DISEASE INCLUDING SYNEILESIS PALMATA EXTRACT AS ACTIVE INGREDIENT, Applicant: Glocal Industry-Academic Cooperation Foundation, Konkuk University, Registration Date: Apr. 26, 2017)
(Patent Document 0002) Korean Patent No. 10-2355586 (Title of the disclosure: PREVENTIVE OR THERAPEUTIC COMPOSITION FOR CRANIAL NERVE DISEASE CONTAINING SYNEILESIS ACONITIFOLIA AS ACTIVE INGREDIENT, Applicant: Lee Il Soo, Registration Date: Jan. 21, 2022)

### SUMMARY OF THE INVENTION

An objective of the present disclosure is to provide an immunity-enhancing composition containing a Syneilesis palmate extract as an active ingredient.

The present disclosure relates to an immunity-enhancing composition containing a Syneilesis palmate extract as an active ingredient.

The Syneilesis palmate extract may be an extract of a whole plant, a leaf, a stem, a root, a fruit, or a flower of Syneilesis palmate.

The Syneilesis palmate extract may be extracted by extracting Syneilesis palmate using water, alcohol having 1 to 4 carbon atoms, or a mixed solution thereof as a solvent.

When preparing the extract of the present disclosure, a filtrate may be dried and powdered. The powderization may be performed by existing drying methods, such as freeze drying, hot air drying, spray drying, and the like.

The Syneilesis palmate extract is characterized by promoting the production of immunomoulatory factors or inducing phagocytosis activation in macrophages.

The Syneilesis palmate extract is an immunomoulatory factor, which induces the production of nitric oxide (NO), nitric oxide synthase (iNOS), interleukin-1β (IL-1β), or tumor necrosis factor-α (TNF-α). In this case, when treating the Syneilesis palmate extractin a concentration of 25 μg/mL NO, the amount of iNOS, IL-1β, or TNF-α is increased by 4 to 5 times, 4 to 6 times, 5 to 10 times, and 2 to 3 times, respectively.

The Syneilesis palmate extract induces macrophage phagocytosis. When treating the Syneilesis palmate extract in a concentration of 25 μg/mL, phagocytosis activity is preferably increased by 2 times or more, and more preferably, by 2 to 3 times.

An effective concentration of the Syneilesis palmate extract is preferably in a range of 12.5 μg/mL to 200 μg/mL to be used as an immunity-enhancing composition.

Hereinafter, the present disclosure will be described in detail.

The Syneilesis palmate extract may be extracted by extracting Syneilesis palmate using water, alcohol having one to four carbon atoms, or a mixed solution thereof as a solvent. The alcohol having one to four carbon atoms may be selected from the group consisting of methanol, ethanol, propanol, isopropanol, butanol, and isobutanol. The solvent is preferably a mixed solvent of water or the alcohol having one to four carbon atoms. The mixed solvent may be an alcohol aqueous solution extract having one to four carbon atoms in an amount of 30% to 90% (v/v), and preferably, in an amount of 50% to 80% (v/v). Among the alcohol having one to four carbon atoms, ethanol may be used. Among the solvents, water is the most preferable. Water, the alcohol having one to four carbon atoms, or the mixed solution thereof, used when preparing the Syneilesis palmate extract, may be used in 1 to 40 times the volume (1 L to 40 L based on 1 kg), and preferably, in 5 to 40 times the volume, with respect to the weightof the Syneilesis palmate extract being used. The Syneilesis palmate extract may be extracted under conditions ofa temperature in a range of 20° C. to 100° C. for 1 minute to 48 hours. The above extraction process may be repeated 1 to 4 times.

Hence, the extract of the present disclosure can be applied to immunity-enhancing food compositions, health supplements, feed compositions for companion animals, a variety of pharmaceutical compositions, and the like.

In addition, the extract of the present disclosure may be prepared as a fraction by conventional methods in the art, in which the extract is dissolved in water, and then additionally fractioned by using at least one solvent selected from the group consisting of n-hexane, methylene chloride, acetone, chloroform, ethyl acetate, and n-butanol.

A conventional extraction device, an ultrasonic pulverization extractor, or a fractionator may be used as a device for extracting the Syneilesis palmata extract or the fraction thereof. Such prepared Syneilesis palmata extract may be hot air-dried, vacuum-dried, or freeze-dried to remove the solvent. In addition, the Syneilesis palmata extract or the fraction thereof may be purified by column chromatography and used.

The Syneilesis palmata extract may be used through fractionation or purification by well-known methods used for separation and extraction of plant components, such as extraction with organic solvents (for example, alcohol, ether, acetone, and the like), hexane and water partitioning, and column chromatography, and the like. The methods may be used solely or in combination.

The chromatography may be selected among silica gel column chromatography, LH-20 column chromatography, ion exchange resin chromatography, medium-pressure liquid chromatography, thin-layer chromatography (TLC), silica gel vacuum liquid chromatography, and high-performance liquid chromatography.

In addition, the present disclosure provides a pharmaceutical composition containing a Syneilesis palmata extract. The Syneilesis palmata extract may be added to the pharmaceutical composition of the present disclosure in an amount of 0.001% to 100% by weight.

The pharmaceutical composition may be used by being formulated into oral formulations, such as powder medicines, granules, tablets, capsules, suspensions, emulsions, syrups, aerosols, and the like, external preparations, suppositories, and sterile injection solutions, according to conventional methods. Examples of carriers, excipients, and diluents that may be contained in the pharmaceutical composition may include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil. When being formulated, excipients or diluents, such as commonly used fillers, extenders, binders, wetting agents, disintegrating agents, and surfactants may be used for compounding. Examples of solid formulations for oral administration include tablets, pills, powder medicines, granules, capsules, and the like. Such solid formulations are compounded by mixing at least one type of excipient, for example, starch, calcium carbonate, sucrose or lactose, gelatin, and the like in the Syneilesis palmata extract. In addition, other than simple excipients, lubricants, such as magnesium stearate and talc, are also used. Examples of liquid formulations for oral administration include suspensions, solutions for internal use, emulsions, syrups, and the like. Other than commonly used diluents, such as water and liquid paraffin, a variety of excipients, for example, wetting agents, sweeteners, odorants, preservatives, and the like, may be included. Examples of formulations for parenteral administration include sterilized aqueous solutions, non-aqueous solvents, suspensions, emulsions, freeze-dried formulations, and suppositories. Examples of the non-aqueous solvents and the suspensions may include propylene glycol, polyethylene glycol, vegetable oils such as olive oil, injectable esters such as ethyl oleate, and the like. Examples of a suppository base may include witepsol, macrogol, tween 61, cacao fat, laurin fat, glycerogelatin, and the like.

A dosage of the pharmaceutical composition of the present disclosure may vary depending on the age, sex, and weight of the subject to be treated, the specific disease or pathological condition to be treated, the severity of the disease or pathological condition, administration route, and the decision of a prescriber. Dosage determination based on such factors falls within the level of those skilled in the art. Generally, the pharmaceutical composition is administered in a dosage range of 0.01 mg/kg/day to approximately 2000 mg/kg/day. More preferably, the pharmaceutical composition is administered in a dosage range of 1 mg/kg/day to 500 mg/kg/day. Administration may be performed once or multiple times a day. The dosage is not intended to limit the scope of the present disclosure in any way.

Mammals, such as rats, livestock, and humans, may be administered the pharmaceutical composition of the present disclosure through various routes. All methods of administration can be envisaged, and may include oral administration, rectal administration, intravenous administration, intramuscular injection, subcutaneous injection, intrauterine injection, and intracerebroventricular injection. The extract of the present disclosure has little toxicity and side effects, and thus is a drug that can be safely used even when taken for a long period of time for preventive purposes.

In addition, the present disclosure provides an immunity-enhancing health supplement containing a Syneilesis palmata extract and a sitologically acceptable food additive. The Syneilesis palmata extract may be added to the health supplement of the present disclosure in an amount of 0.001% to 100% by weight. The health supplement of the present disclosure may be provided in the form of tablets, capsules, pills or liquids. Examples of the health supplement to which the extract of the present disclosure can be added may include a variety of drinks, meat, sausages, bread, candy, snacks, noodles, ice cream, dairy products, soups, ionic beverages, soft drinks, alcoholic beverages, chewing gum, tea, vitamin complexes, and the like.

The present disclosure relates to an immunity-enhancing composition containing a Syneilesis palmata extract as an active ingredient.

The Syneilesis palmata extract activates macrophage phagocytosis, promotes the production of nitric oxide (NO), inducible nitric oxide synthase (iNOS), IL-1β (interleukin-1β), and tumor necrosis factor-α (TNF-α), and induces TLR2/4 stimulation. In addition, the Syneilesis palmata extract exhibits immunity-enhancing activity by activating macrophage autophagy, suggesting that the Syneilesis palmata extract can be applied to a variety of health supplements.

### BRIEF DESCRIPTION OF THE DRAWINGS

FIGS. 1A and 1B are diagrams showing the results of confirming NO production and gene expression of inducible nitric oxide synthase (iNOS), interleukin-1β (IL-1β), and tumor necrosis factor-α (TNF-α) by treating macrophages with a Syneilesis palmata extract (SPL) at different concentrations;

FIG. 2 is a diagram showing the results of inducing phagocytosis activation by treating macrophages with a Syneilesis palmata extract (SPL) at different concentrations;

FIGS. 3A, 3B and 3C are diagrams showing the results of confirming the induction of NO production and gene expression of iNOS, IL-1β, and TNF-α by treating a Syneilesis palmata extract (SPL) with a TLR2/4 inhibitor in macrophages and then allowing the extract to strongly induce TLR4 expression (TLR2 expression indicates a low response);

FIGS. 4A, 4B, 4C and 4D are diagrams showing the results of confirming NO production and gene expression of inducible nitric oxide synthase (iNOS), interleukin-1β (IL-1β), and tumor necrosis factor-α (TNF-α) after treating macrophages with a MAPK inhibitor to inhibit a variety of signaling pathways, in which p38 is confirmed to be the main upstream kinase involved in the production of immunity-enhancing factors due to a Syneilesis palmata extract (SPL); and

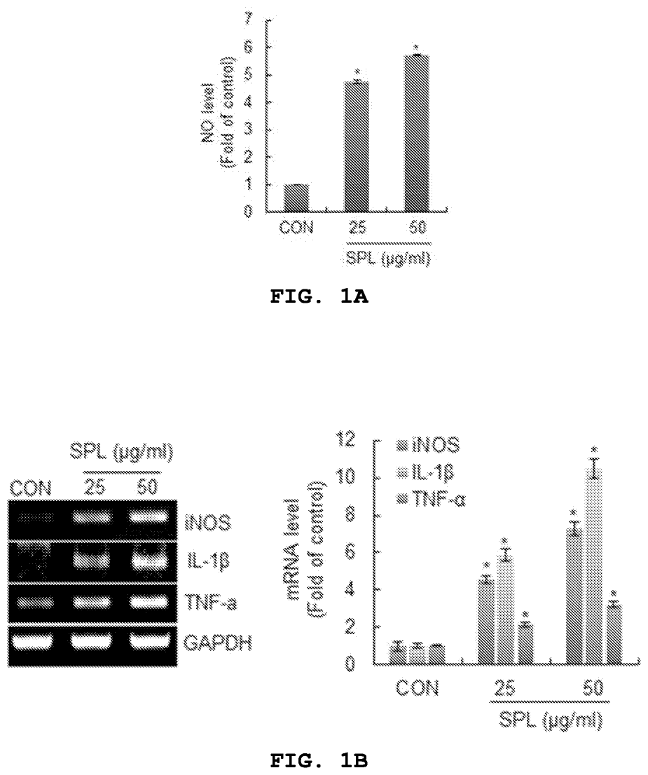

FIGS. 5A, 5B and 5C are diagrams showing the results of confirming that a Syneilesis palmata extract (SPL) phosphorylates p38 in macrophages and induces p38 activation using TLR2/4 through protein expression, indicating that macrophage autophagy is activated through TLR2/4 and p38 activation.
In each drawing of FIGS. 1A to 5C, the results of gene/protein expression were digitized and graphed.

### DESCRIPTION OF THE PREFERRED EMBODIMENTS

Hereinafter, preferred embodiments of the present disclosure will be described in detail. However, the present disclosure is not limited to the embodiments described herein and may be embodied in other forms. Rather, the embodiments are provided to sufficiently convey the spirit of the disclosure to those skilled in the art, so that the disclosure herein will be thorough and complete.

### Example 1: Preparation of Syneilesis palmata Extract

To prepare a Syneilesis palmata extract of the present disclosure, water was used as an extraction solvent. Specifically, Syneilesis palmata leaves were washed with distilled water 2 to 3 times, dried with a hot-air dryer, ground with a grinder, and stored at a temperature of −20° C. Water was added to the ground Syneilesis palmata leaves at an amount equivalent to the 20-fold volume of 10 g of the ground Syneilesis palmata leaves. Then the ground leaves were extracted at a temperature of 60° C. for 3 hours, and the extract was obtained. Thereafter, the extract was filtered and lyophilized to obtain the final Syneilesis palmata extract (SPL).

### Example 2:Confirmation of Cell Culture and Cytotoxicity of Syneilesis palmata Extract

Mouse macrophage RAW264.7 was purchased from the American Type Culture Collection (Manassas, VA, USA) and cultured using a culture medium prepared by mixing a 10% fetal bovine serum (purchased from Hyclone Laboratories, Logan, UT, USA), 100 U/ml of penicillin, and 100 μg/ml of streptomycin (purchased from Gibco BRL, Grand Island, NY, USA) in Dulbecco's modified eagle medium (DMEM)/F-12 1:1 modified medium (purchased from Lonza, Walkersville, MD, USA) at a temperature of 37° C. with 5% CO₂.

A Syneilesis palmata extract (SPL) was dissolved in sterile water and added to the cells before the experiment.

On the other hand, the cytotoxicity evaluation was performed with the MTT assay that utilizes the principles that dehydrogenases in the mitochondria of cells with intact metabolic processes reduce yellow water-soluble tetrazolium salt [3-(4,5-dimethylthiazol-2-yl)-2-5-diphenyltrazolium bromide] (MTT) to non-soluble dark purple MTT formazan crystals. The cytotoxicity evaluation was performed on the crystals by measuring absorbance at an appropriate wavelength (mainly in a range of 500 nm to 600 nm).

First, the RAW 264.7 cells were dispensed into a 96-well plate at a concentration of 1×10⁵ cells/well, cultured for 24 hours, and then treated with the extract under conditions at concentrations of 0 μg/mL, 50 μg/mL, 100 μg/mL, and 200 μg/mL. After culturing for 24 hours, MTT was added at a concentration of 1 mg/mL, and the cultured cells underwent reactions in an incubator at a temperature of 37° C. for 2 hours. Then, dimethyl sulfoxide (DMSO) was added, and absorbance was measured at a wavelength of 570 nm using a microplate reader.

As a result, it was confirmed that 95% or more of the cells survived with the Syneilesis palmata extract (SPL) prepared in Example 1, indicating no cytotoxicity was exhibited.

### Example 3: Measurement of Syneilesis palmata Extract Activity Inducing Production of Immunostimulatory Factors

Griess assay and reverse transcription-polymerase chain reaction (RT-PCR) analysis were performed to confirm whether the production of immunomodulatory factors, nitric oxide (NO), inducible nitric oxide synthase (iNOS), interleukin-1 beta (IL-1β), and tumor necrosis factor alpha (TNF-α), were promoted due to Syneilesis palmata extract (SPL).

To perform Griess assay for NO production measurement, RAW264.7 cells (2×10⁵ cells/well) were first cultured in a 12-well plate for 24 hours, treated with the SPL, and then additionally cultured for 24 hours. Thereafter, the cultured cells were mixed with a Griess reagent (purchased from Sigma Aldrich) in a 100-μl cell culture medium, and underwent reactions at room temperature for 15 minutes. After the reactions, absorbance was measured at a wavelength of 540 nm using a UV/Visible spectrophotometer (purchased from Human Cop., Xma-3000PC, Seoul, Korea).

To evaluate the effect of the SPL on the expression of iNOS, IL-1β, and TNF-α, RT-PCR analysis was performed. To this end, RAW264.7 cells (2×10⁵ cells/well) were cultured in a 12-well plate for 24 hours, treated with the SPL, and additionally cultured for 24 hours. After all of the processes were complete, total RNAs were removed from the cells using a RNeasy mini kit (purchased from Qiagen, Valencia, CA, USA), and the total RNAs were quantitatively analyzed. Thereafter, cDNA was synthesized from 1 μg of the total RNAs using a Verso cDNA kit (purchased from Thermo Scientific, Pittsburgh, PA, USA). Then, PCR was performed using a PCR master mix kit (purchased from Promega, Madison, WI, USA) and primers presented in Table 1. The results of PCR were visualized using agarose gel electrophoresis. mRNA band density was measured using the UN-SCAN-IT gel software version 5.1 (purchased from Silk Scientific Inc., Orem, UT, USA).

TABLE 1

| Primer name | Base sequence |
|---|---|
| iNOS F | 5′-ttgtgcatcgacctaggctggaa-3′ |
| iNOS R | 5′-gacctttcgcattagcatggaagc-3′ |
| IL-1β F | 5′-ggcaggcagtatcactcatt-3′ |
| IL-1β R | 5′-cccaaggccacaggtattt-3′ |
| TNF-α F | 5′-tggaactggcagaagaggca-3′ |
| TNF-α R | 5′-tgctcctccacttggtggtt-3′ |
| GAPDH F | 5′-ggactgtggtcatgagcccttcca-3′ |
| GAPDH R | 5′-actcacggcaaattcaacggcac-3′ |

As shown in the results of FIGS. 1A and 1B, the SPL promoted the production of immunostimulatory factors, NO, iNOS, IL-1β, and TNF-α, in a concentration-dependent manner.

### Example 4: Measurement of the Effect of Syneilesis palmata Extract on Macrophage Phagocytosis

Immunomodulator secreted from macrophages are known to promote macrophage phagocytosis, and macrophage phagocytosis is used as an indicator of macrophage activation. Therefore, a neutral red uptake assay was performed to evaluate the effect of Umbrella extract (SPL) on phagocytosis of RAW264.7 cells.

Specifically, the RAW264.7 cells (2×10⁵ cells/well) were cultured in a 12-well plate for 24 hours, treated with the SPL by varying concentrations, and additionally cultured for 24 hours. Each of the cells was washed three times with 1×PBS after 24 hours. Then, 1 ml of a 0.01% neutral red solution was added to each of the cells and cultured for 2 hours. Thereafter, each of the cells was washed three times with 1×PBS, and then, a 1-ml cell lysis buffer (ethanolic acid:acetic acid=1:1) was added to elute the neutral red solution absorbed by the cells. Absorbance was measured at a wavelength of 540 nm using a UV/Visible spectrophotometer (purchased from Human Cop., Xma-3000PC, Seoul, Korea).

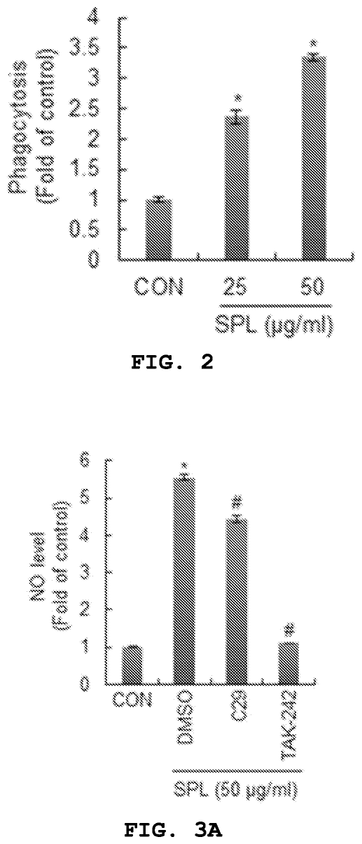

As shown in the results of FIG. 2, it was found that the SPL significantly promoted macrophage phagocytosis.

Therefore, considering the results of FIGS. 1A, 1B and 2, the fact that the SPL promotes the production of the immunostimulatory factors and the macrophage phagocytosis may prove that the SPL is a composition with immunity-enhancing activity, which can induce macrophage phagocytosis.

### Example 5: Identification of Receptors Related to Macrophage Activation Induced by Syneilesis palmata Extract

A Syneilesis palmata extract (SPL) of the present disclosure was confirmed to induce macrophage activation, so the main receptors related to macrophage activation were identified to track the mechanism.

To this end, the effect of Toll-like receptor 2/4 (TLR2/4), widely known as the main receptor associated with macrophage activation, on the production of immunomodulator induced by the SPL was evaluated.

Specifically, RAW264.7 cells were treated with the SPL for 24 hours after inhibiting TLR2 or TLR4 with C29 (TLR2 inhibitor) or TAK-242 (TLR4 inhibitor). NO levels were analyzed by Griess assay, and NOS, IL-1β, and TNF-α were analyzed by RT-PCR. In addition, phagocytosis was measured by neutral red uptake assay.

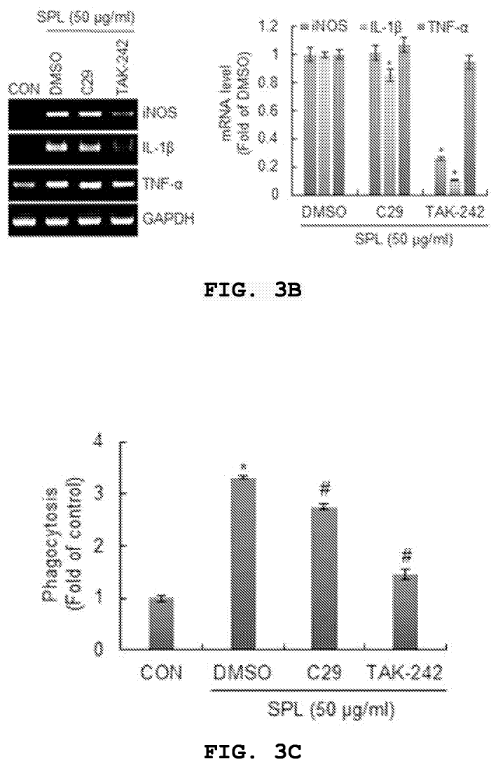

As shown in the results of FIG. 3A, it was confirmed that the SPL inhibited by C29 still slightly affected the immunity-related factors, such as NO and IL-1β. In addition, as shown in FIG. 3B, TAK-242 inhibition of the SPL activity significantly decreased the production of NO, iNOS, and IL-1β.

In addition, as shown in FIG. 3C, macrophage phagocytosis enhanced by the SPL also slightly affected C29, and TLR2 was thus slightly inhibited while TLR4 inhibition by TAK-242 was strong.

Therefore, it is seen that the main macrophage receptor related to macrophage activation by the SPL is TLR4. That is, it is confirmed that the SPL slightly induces Toll-like receptor 2 (TLR2) expression in the macrophages, and mainly induces Toll-like receptor 4 (TLR4) expression to promote the production of the immunomodulators.

### Example 6: Identification of Upstream Kinases Involved in the Production of Immunomodulators Induced by Syneilesis palmata Extract (SPL)

TLR4 activation is known to activate signaling pathways of mitogen-activated protein kinase (MAPK) to induce the production of immunomodulators in macrophages. Therefore, whether the MAPK signaling was involved in the production of Syneilesis palmata extract (SPL)-mediated immunomodulator in the macrophages was analyzed.

Specifically, before being treated with the SPL,RAW264.7 cells were pretreated with PD98059 (40 μM), an ERK1/2 inhibitor, SB203580 (40 μM), a p38 inhibitor, and SP600125 (40 μM), a JNK inhibitor to inhibit respective signaling pathways, and then treated with the SPL for 24 hours. NO levels were analyzed by Griess assay, and NOS, IL-1β, and TNF-α were analyzed by RT-PCR.

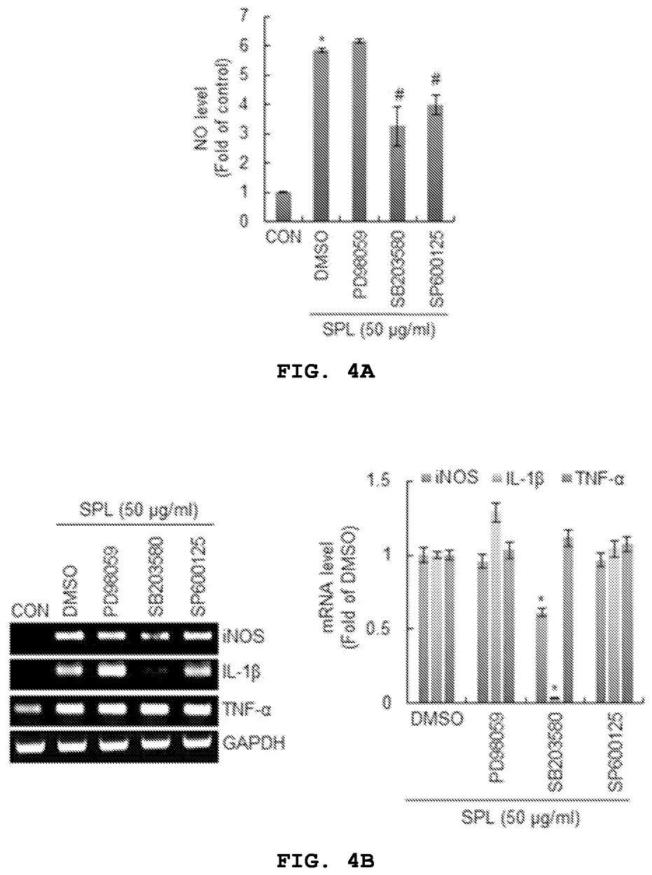

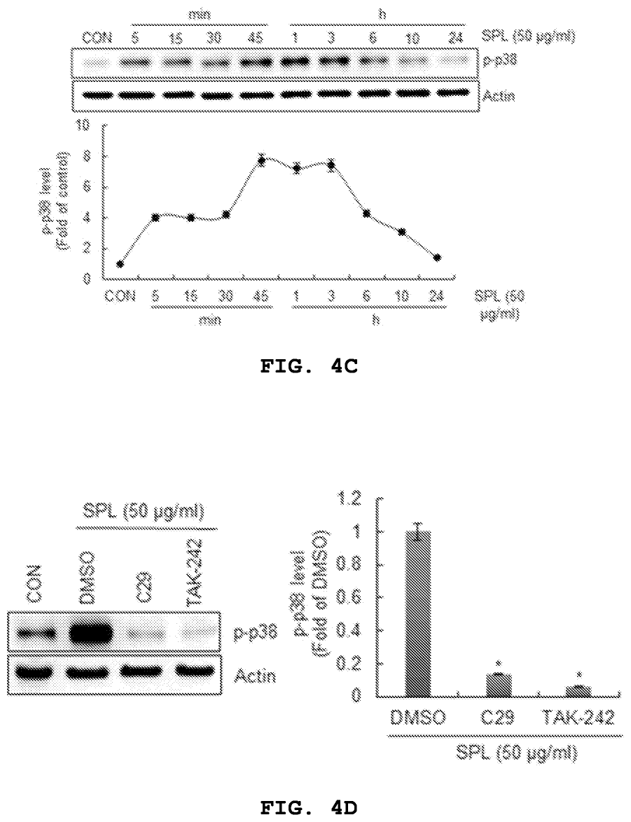

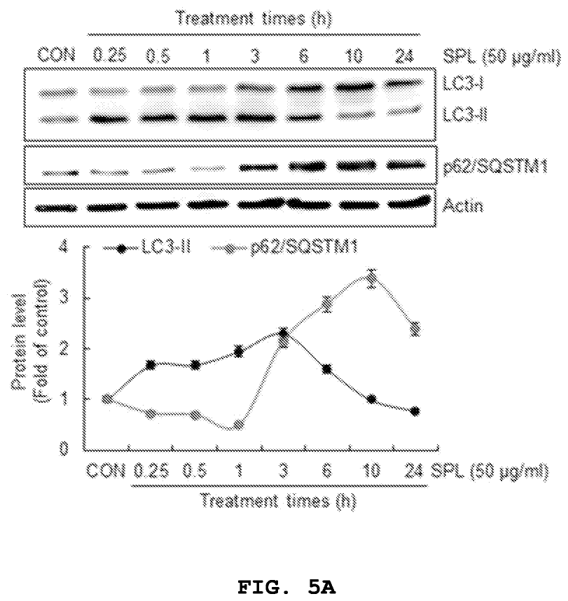

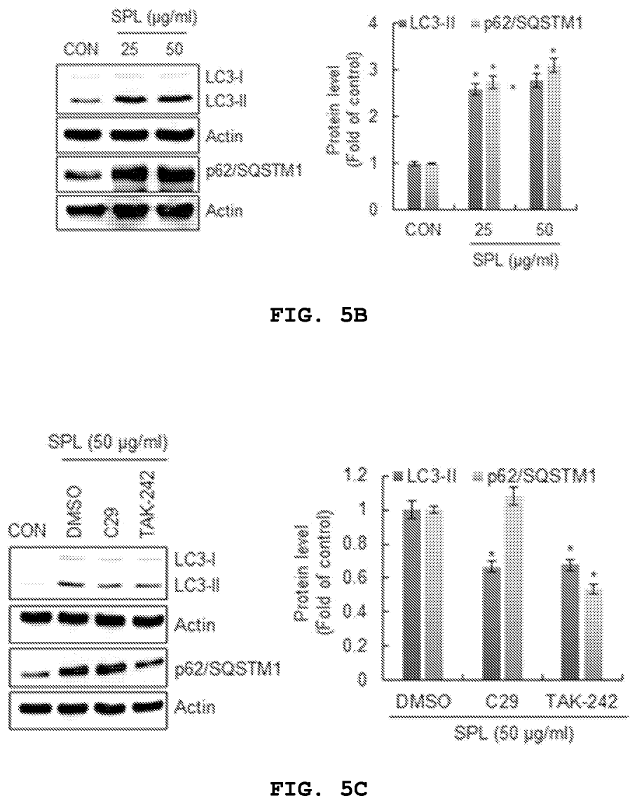

As shown in the results of FIGS. 4A and 4B, the SPL induced the production of NO, NOS, IL-1β, and TNF-α in the macrophages regardless of whether ERK1/2 signaling was inhibited or not. In addition, JNK signaling inhibition only decreased the NO production induced by SPL, but the expression of other factors was maintained.

On the other hand, when inhibiting p38 signaling, an increase in the production of NO, iNOS, and IL-1β expressed by SPL induction was reduced. As a result, it is confirmed that p38 is the most important upstream kinase involved in the production of the immunostimulatory factors induced by SPL. ※However, SPL-induced TNF-α expression had no effect on p38 inhibition and JNK inhibition.

Next, to analyze the effect of the SPL on p38 activation, RAW264.7 cells were treated with the SPL by time, and then p38 phosphorylation was examined by Western blot analysis.

For the experiment, the SPL-treated cells were washed three times with cold 1×PBS, and then added to a radioimmunoprecipitation (RIPA) buffer solution (purchased from Boston Bio Products, Ashland, MA, USA) containing a protease inhibitor (purchased from Sigma Aldrich) and phosphatase inhibitors (purchased from Sigma Aldrich) to undergo reactions at a temperature of 4° C. for 30 minutes. The cells that reacted with the RIPA buffer solution were centrifuged at 15,000 rpm at a temperature of 4° C. for 10 minutes to obtain the supernatant. Then, the proteins were quantitatively analyzed using a bicinchoninic acid (BCA) protein assay kit (purchased from Thermo Fisher Scientific, Waltham, MA, USA).

Thereafter, the proteins were removed from SDS-PGAE and transferred to a polyvinylidene difluoride (PVDF) membrane. The PVDF membrane underwent reactions for 1 hour in a blocking buffer (0.05% Tween 20 (TBS-T) containing 5% skim milk) at room temperature. Next, the PVDF membrane was washed with a TBS-T buffer, treated with a specific primary antibody in 0.05% TBS-T with 5% BSA, and then left at a temperature of 4° C. for 16 hours. The PVDF membrane was washed with TBS-T buffer after being treated with the primary antibody and then subsequently treated with a secondary antibody by being stirred in a blocking buffer at room temperature. Chemiluminescence was performed using an ECL Western blotting substrate (purchased from Amersham Biosciences, Piscataway, NJ, USA), after washing the PVDF membrane with the TBS-T buffer, and visualized by LI-COR C-DiGit Blot Scanner (purchased from Li-COR Biosciences, Lincoln, NE, USA). Band density in Western blot was measured using the UN-SCAN-IT gel software version 5.1 (purchased from Silk Scientific Inc., Orem, UT, USA).

As shown in the results of FIG. 4C, it is confirmed that the p38 phosphorylation induced by SPL starts 5 minutes after the treatment, which is maximized at 3 hours of the treatment.

Lastly, to analyze the effect of TLR2/4 on SPL-induced p38 activation, C29 and TAK-242 were used to inhibit TLR2 and TLR4, respectively. Then, p38 phosphorylation was examined by Western blot analysis after treating the SPL for 3 hours.

As shown in FIG. 4D, TLR2/4 inhibition reduced SPL-induced p38 phosphorylation. Considering the results of FIG. 4D, it is seen that the SPL induces p38 activation viaTLR2/4 stimulation.

### Example 7: Confirmation of Macrophage Autophagy Induced by Syneilesis palmata Extract

TLR-mediated autophagy has been reported to promote immune responses through enhanced antigen processing and presentation in antigen-presenting cells. In addition, stimulation of autophagy has recently been reported to promote T cell responses by regulating the functions of both antigen-presenting cells and T cells. Therefore, an autophagy promoter is considered a potential candidate for an immunoadjuvant to promote the immune response.

Hence, to confirm whether a Syneilesis palmata extract (SPL) of the present disclosure had an autophagy activation effect, the expression levels of LC3 and p62/SQSTM1 were specified through Western blotting. Conversion of LC3-I to LC3-II is used as a key indicator of autophagy activation. p62/SQSTM1 is also known to promote autophagosome formation by interacting with LC3.

As shown in the results of FIGS. 5A and 5B, an increase in the production of LC3-II started 15 minutes after SPL treatment in macrophages. In addition, an increase in the production of p62/SQSTM1 (Sequestosome1) started 3 hours after the treatment. In addition, the expression of LC3-II and p62/SQSTM1 increased in a concentration-dependent manner with respect to the extract. Through the results, it is confirmed that the SPL induces macrophage autophagy.

In addition, it was confirmed that the SPL activated the macrophages through TLR2/4 signaling. Thus, to confirm the effect of SPL-induced macrophage autophagy on TLR2/4, Western blot analysis was performed.

As shown in the results of FIG. 5C, TLR4 inhibition induced by TAK-242 reduced the production of LC3-II and p62/SQSTM1, SPLP-induced autophagy factors. In addition, TLR2 inhibition induced by C29 reduced the production of 62/SQSTM1. Therefore, it is believed that the SPL stimulates TLR2/4 and thus induces macrophage autophagy.

### Formulation Example 1. Pharmaceutical Formulation

200 g of Syneilesis palmata extract of the present disclosure was mixed with 175.9 g of lactose, 180 g of potato starch, and 32 g of colloidal silicic acid. A 10% gelatin solution was added to the mixture. Then, the resulting mixture was ground and then filtered through a 14 mesh sieve. The filtered mixture was dried, and the mixture obtained by adding 160 g of potato starch, 50 g of talc, and 5 g of magnesium stearate was made into tablets.

### Formulation Example 2. Food Preparation

### Formulation Example 2-1. Preparation of Cooking Seasoning

A Syneilesis palmata extract of the present disclosure was added to a cooking seasoning in an amount of 1% by weight to prepare a health-promoting cooking seasoning.

### Formulation Example 2-2. Preparation of Flour Food

A Syneilesis palmata extract of the present disclosure was added to flour in an amount of 0.1% by weight to prepare a mixture. Then, health-promoting foods, such as bread, cakes, cookies, crackers, and noodles, were prepared with the mixture.

### Formulation Example 2-3. Preparation of Soup and Gravies

A Syneilesis palmata extract of the present disclosure was added to soup and gravy in an amount of 0.1% by weight to prepare health-promoting soup and gravy.

### Formulation Example 2-4. Preparation of Dairy Products

A Syneilesis palmata extract of the present disclosure was added to milk in an amount of 0.1% by weight. Then, dairy products, such as butter and ice cream, were prepared with the milk.

### Formulation Example 2-5. Preparation of Vegetable Juice

0.5 g of a Syneilesis palmata extract of the present disclosure was added to 1000 ml of tomato juice or carrot juice to prepare health-promoting vegetable juice.

### Formulation Example 2-6. Preparation of Fruit Juice

0.1 g of a Syneilesis palmata extract of the present disclosure was added to 1000 ml of apple juice or grape juice to prepare health-promoting fruit juice.

What is claimed is:

1. An immunity-enhancing composition comprising a Syneilesis palmata extract as an active ingredient.

2. The composition of claim 1, wherein the extract promotes production of an immunomoulatory factor or induces phagocytosis activation in a macrophage.

3. The composition of claim 2, wherein the immunomoulatory factor comprises at least one selected from the group consisting of nitric oxide (NO), inducible nitric oxide synthase (iNOS), interleukin-1β (IL-1β) and tumor necrosis factor-α (TNF-α).

4. The composition of claim 1, wherein the extract induces macrophage autophagy.

5. An immunity-enhancing food composition comprising the composition of claim 1.

6. An immunity-enhancing health supplement food comprising the composition of claim 1.

7. An immunity-enhancing feed composition for a companion animal comprising the composition of claim 1.
